(19) United States
(12) Patent Application Publication
(10) Pub. No.: US 2014/0046245 A1
(43) Pub. Date: Feb. 13, 2014

(54) PERCUTANEOUS TRANSPEDICULAR ENDOVERTEBRAL DEVICE

(71) Applicant: Louis Cornacchia, Point Lookout, NY (US)

(72) Inventor: Louis Cornacchia, Point Lookout, NY (US)

(21) Appl. No.: 13/965,898

(22) Filed: Aug. 13, 2013

Related U.S. Application Data
(60) Provisional application No. 61/682,559, filed on Aug. 13, 2012.
(60) Provisional application No. 61/782,847, filed on Mar. 14, 2013.

(51) Int. Cl. A61B 17/16

(52) U.S. Cl. CPC A61B 17/1671
USPC 604/22

(57) ABSTRACT

A percutaneous transpedicular endovertebral device and system is disclosed. An example device includes a cannula having an internal lumen. The device also includes a trocar receivable within the cannula internal lumen. A flexible shaft cutting catheter is receivable through the trocar to form a cavity within a vertebrae structure and/or across intervertebral discs such that the cannuale can span across more than one vertebral body. A delivery tube is receivable through the trocar to deliver filling, biological agent, or other material to the cavity.

### PRIORITY CLAIM

This application claims priority to U.S. Provisional Patent Application No. 61/682,559 filed on Aug. 13, 2012, titled “Percutaneous Transpedicular Endovertebral Intervertebral Kinetic Modulation” of Louis Cornacchia, and U.S. Provisional Patent Application No. 61/782,847 filed on Mar. 14, 2013, titled “Percutaneous Transpedicular Endovertebral Intervertebral Kinetic Modulation” of Louis Cornacchia, each of which is hereby incorporated by reference for all that is disclosed as though fully set forth herein.

### BACKGROUND

Vertebra includes an anterior portion (or vertebral body) bearing most of a body's weight, and a posterior portion including the lamina and the spinous processes. A pedicle connects the anterior portion with the posterior portion with a column of bone. Intervertebral space (or discs) separate adjacent vertebra and facilitate movement or articulation between adjacent vertebrae.

A herniated lumbar disc is a condition where material bulges from the intervertebral space between the vertebral bodies and into the spinal canal or neural foramina. This may result in nerve root compression, spinal cord compression or both, and may cause pain and neurological deficit including, in some instances, paralysis.

### BRIEF DESCRIPTION OF THE DRAWINGS

FIG. 1 illustrates an example medical device.

FIG. 1A illustrates another example of an instrument for use with the medical device.

FIG. 1B is a plot illustrating example strain results for a Nitinol™ tube being straightened from a 90-degree angle.

FIG. 2 illustrates example flexible access guides which may be implemented with the device.

FIG. 3 illustrates example flexible shaft cutting blades which may be implemented with the device.

FIG. 4 illustrates example flexible shaft balloon catheters which may be implemented with the device.

FIGS. 5A-C illustrate another example of a flexible shaft catheter having a balloon tip.

FIGS. 6A-B illustrate another example cutting tip which may be used with the medical device.

FIG. 7 illustrates an example flexible shaft suction tubing which may be implemented with the device.

FIG. 8 illustrates example flexible shaft probes which may be implemented with the device.

FIG. 9 illustrates example fill tubes which may be implemented with the device.

FIGS. 10A-B illustrate other example catheter tips which may be provided on any of the trocar, cannula, catheter, tubing, probes, and/or other bodies described herein, to provide direction.

FIG. 11 illustrates example endovertebral spinal surgery techniques.

FIGS. 12A-B and FIGS. 13A-B illustrate side and top views of the medical device during endovertebral spinal surgery.

FIGS. 14A-B illustrate side and top views of example insertion of balloon into vertebrae to create a cavity in the vertebrae area.

FIGS. 15A-B illustrate side and top views of example insertion of cutting guide into vertebra.

FIGS. 16A-B illustrate side and top views of example flexible shaft cutting blades passing through a cutting guide which is received within a cannula.

FIGS. 17A-B illustrate side and top views of example use of a device to core out a cylinder in the vertebral bodies, and across disc space.

FIGS. 18A-B illustrate example filling of a cavity.

### DETAILED DESCRIPTION

Spinal fusion is an example of a corrective measure for treating low back pain. During the procedure, support is provided between vertebrae adjacent to the herniated disc to space the discs apart from one another and restrict or altogether eliminate relative motion of the vertebrae.

Spinal fusion surgery may be performed using open surgery techniques, or by minimally invasive techniques that access the spine through small percutaneous pathways. Successful minimally invasive procedures can shorten hospital stays and recovery time, as well as perioperative pain. Unfortunately, some minimally invasive spine surgery techniques can be awkward for the surgeon to perform, and sometimes can be more time-consuming than open surgery.

For example, a common technique is for the surgeon to implant screws from the posterior aspect of the spine through the pedicle into vertebral body under fluoroscopic guidance. Then the surgeon uses an arc-geometry device to implant a connecting bar across the implanted screws. This procedure can take more than several hours. In addition, the learning curve for the surgeon can be significant, and the outcome may be similar to outcomes that can be achieved using conventional open procedures. In addition, this technique does not allow for posterolateral fusion, and a separate procedure is often needed which uses a larger incision to achieve interbody fusion.

Examples of a device (e.g., a medical device) are disclosed herein which enable Percutaneous Transpedicular Endovertebral Inter-vertebral Modulation (“PTEIM”), also referred to herein as “Endovertebral Spinal Surgery” (ESS) or “Endovertebral Surgery” for short. In an example, the device may be provided as part of a percutaneous transpedicular endovertebral system or “kit” including different components which can be used interchangeably depending at least in part on the procedure, the patient and/or preference of the surgeon.

The medical device enables surgical access to the weight-bearing portion of the spine percutaneously through the pedicle. The medical device described herein expands the range of fixation modulus that can be achieved between adjacent vertebrae in bony fusion to restore spinal anatomy. At one end of the spectrum, this would result in fusion of the two vertebral body such that there is no movement between the two vertebral bodies. At the other end of the spectrum, this would result in restriction of movement in one or multiple planes. Of course the device may be used for these and/or other example procedures.

As such, the medical device described herein provides a better overall approach to modifying the relationship between adjacent vertebral bodies, without altering the surrounding anatomy, thereby reducing the time a patient and surgeon have to spend in surgery, and reducing or altogether eliminating hospitalization following the procedure.

It is noted that the medical device described herein may be used for percutaneous pedicle access for spinal fusion, as well as vertebroplasty and kyphoplasty surgeries. For example, the medical device may be used according to endovertebral spinal surgery techniques described herein to access the disc space and remove disc material through a trocar without having to make any incisions at all.

Before continuing, it is noted that as used herein, the terms “includes” and “including” mean, but is not limited to, “includes” or “including” and “includes at least” or “including at least.” The term “based on” means “based on” and “based at least in part on.”

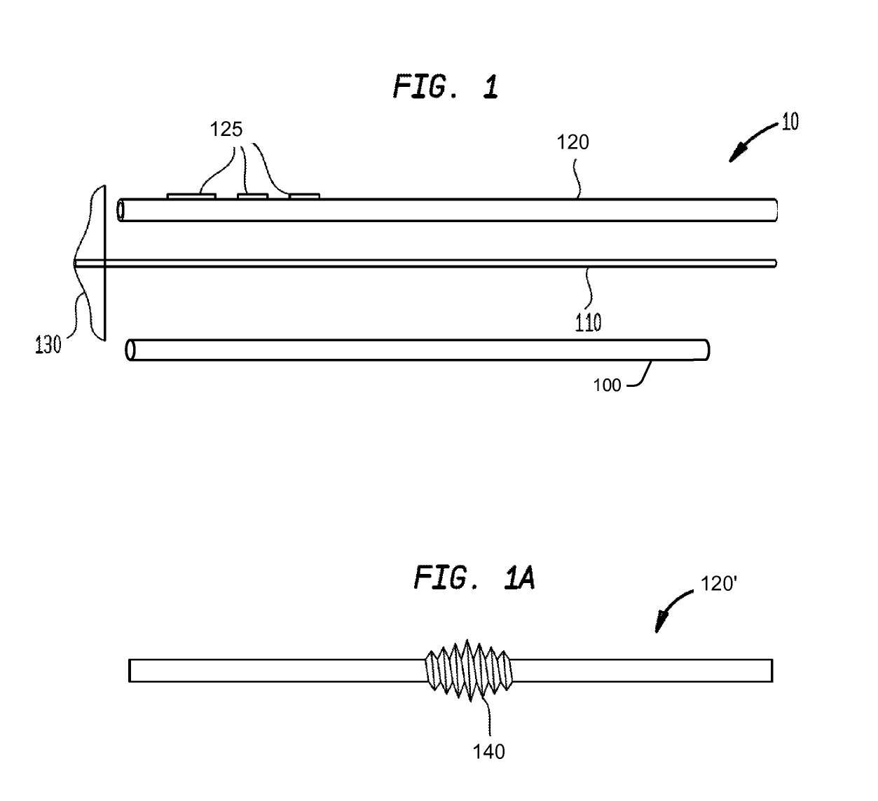

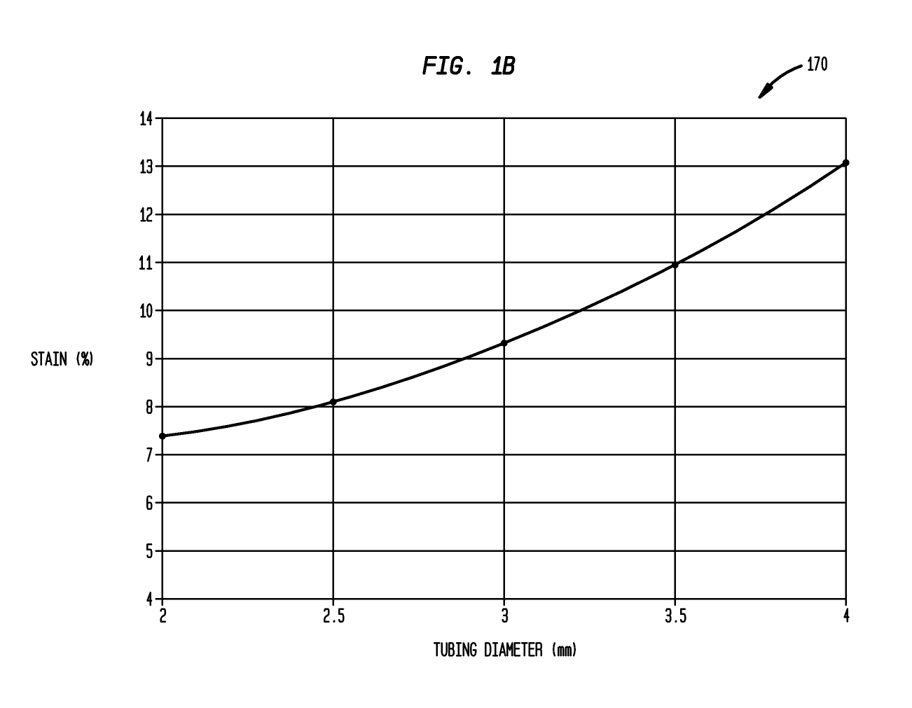

FIG. 1 illustrates an example medical device 10. The medical device 10 may include a cannula 100, a trocar 110, and an instrument 120. Instrument 120 may be provided for inserting the cannula 100 through the skin and muscle and then through a pedicle to gain access to a vertebral body.

The cannula 100 may have an internal lumen of a diameter sized to accommodate the outer diameter of the trocar 110. The trocar 110 may have an outer diameter smaller than the diameter of the cannula internal lumen and is receivable within the cannula 100 by insertion of a distal end of the trocar 110 into a proximal end of the cannula 100. Trocar 110 may be slid through the cannula 100, e.g., until handle 130 abuts the proximal end of cannula 100, to form a cannula and trocar assembly. Once trocar 110 is inserted into 100, both may be inserted through a 3 mm skin incision through soft tissues of the back, and ultimately through the pedicle and into the desired vertebral body.

In an example, the instrument 120 may include at least one small flange 125 at its exterior surface. The flange(s) 125 serve as points of attachment for external fixation of the catheter so that it can be immobilized while the rest of the procedure is performed through the cannula.

FIG. 1A illustrates another example of an instrument 120′ for use with the medical device. This example includes a segment with an external thread 140 that can be used to secure the cannula to the pedicle so as to restrict movement while procedures are performed through the cannula. In an example, the thread profile is tapered. In another example, the thread profile tapers in two directions (as shown in FIG. 1A).

The instrument 120′ is a rigid rod which can be inserted by first inserting a trocar into the cannula, then using a mallet to tamp the instrument 120′ into the pedicle. In an example, the instrument 120′ can then be turned (e.g., in a clockwise manner) with forward pressure to insert the tapered threading into a bone structure. While in the bone structure, the threading provides a firm mount for the instrument 120′ and hence the cannula. The instrument 120′ can be removed by turning (e.g., in a counter-clockwise manner) to remove the tapered threaded from the bone structure.

With reference again to FIG. 1, trocar 110 is capable of both distorted and remembered shapes. As an example, trocar 110 may have a straight profile in a distorted shape and a curved or angled profile in the remembered shape. As such, an angled trocar 110 can be passed through a straight cannula 100 in the distorted (e.g., straightened) shape and then assume an original (e.g., angled) shape upon exiting a distal end of the cannula 100. In some examples, trocar 110 may be configured to convert between distorted and remembered shapes upon induction of temperature changes and may exhibit either one-way or two-way shape memory effects.

Any of a variety of materials may be chosen to manifest this characteristic including but not limited to shape-memory materials (e.g., Nitinol™). The characteristics of Nitinol™ is highly dependent upon conditions of forming and processing, and therefore various strains may be achievable. FIG. 1B is a plot 170 illustrating example strain results for a Nitinol™ tube being straightened from a 90-degree angle. It can be seen from the plot 170, that the elastic limit for the Nitinol™ strain is about 8%. A tubing diameter of 3 mm has a strain of about 9%, and so there may be some (albeit minimal) plastic deformation. A tubing diameter of 2.5 mm may not have any permanent deformation upon being straightened and released back to a remembered state. Of course, the medical device 10 is not limited to manufacture of any particular type(s) of material and/or sizing.

In an example, medical device 10 may be configured for insertion, percutaneously, through a small skin incision through a pedicle and further into a vertebral body and may be used as part of a system to perform percutaneous endovertebral surgery. Following insertion of the cannula and trocar assembly, various catheter devices may be accepted for performing a spinal surgery. Example catheter devices will now be described.

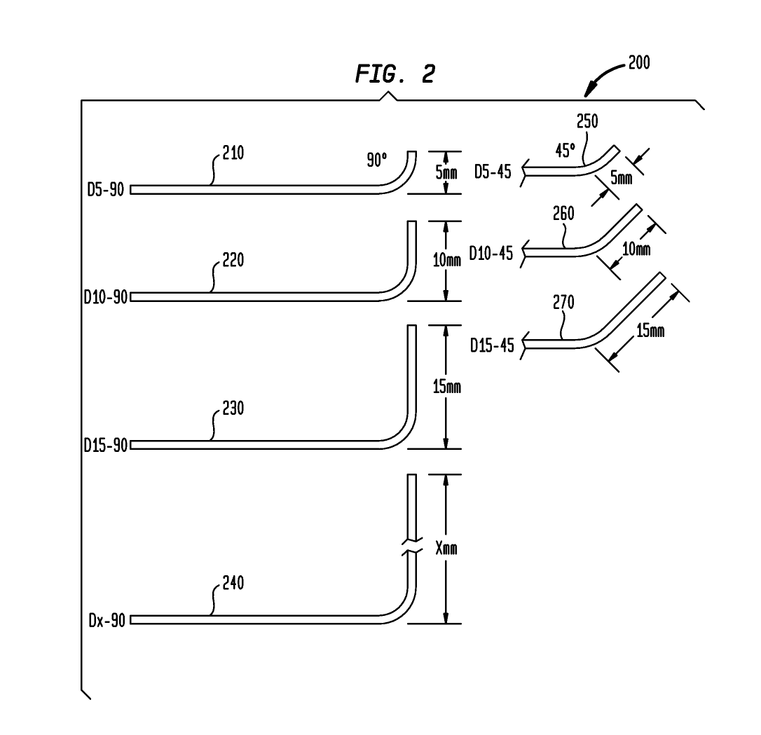

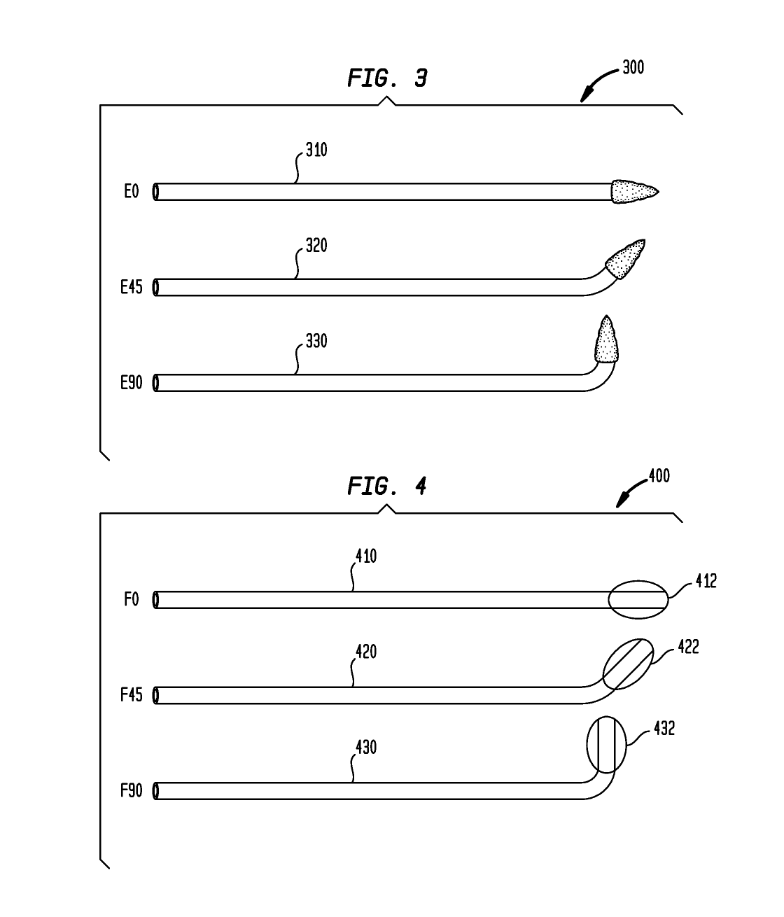

FIG. 2 illustrates example flexible access guides 200 (individually referenced as 210-240 and 250-270) which may be implemented with the medical device 10 shown in FIG. 1. Each flexible access guide (or cutting guide) 200 may include an internal lumen extending between proximal and distal ends. The diameter of the internal lumen may be selected to accommodate instruments such as cutting tips or “drill bits” (see FIG. 3), while the outer diameter of the cutting guide 200 may be selected so that the cutting guide 200 can be accommodated within a cannula (e.g., cannula 100).

As with trocar 110, cutting guide 200 may also be capable of having both distorted and remembered states. That is, a curved or angled cutting guide 200 can be deformed (e.g., to a substantially straight configuration) for insertion into cannula and trocar assembly (also having a straight profile), and then emerge from the distal end of cannula and trocar assembly in a remembered configuration (e.g., the original curved or angled profile). Again, any of a variety of materials may be chosen to manifest this characteristic including but not limited to shape memory metals such as Nitinol™. In some examples, cutting guide 200 may be configured to convert between distorted and remembered shapes upon induction of temperature changes and may exhibit either one-way or two-way shape memory effects.

The cutting guides 210-240 are shown as each may have a distal end which extends at approximately 90 degrees relative to the body or shaft of the drill guide in the remembered state. The cutting guide 210-240 are also illustrated having different diameters. For example, cutting guide 210 is a D5-90 (e.g., a 5 mm diameter cutting guide with a 90 degree bend at the distal end).

The distal end of each of the cutting guides 210-240 is shown having varying lengths. For example, the distal end of cutting guide 210 is illustrated as 5 mm; the distal end of cutting guide 220 is illustrated as 10 mm; and the distal end of cutting guide 230 is illustrated as 15 mm. However, the distal ends may be any of a variety of lengths based on practical application, as illustrated by distal end of cutting guide 240.

The cutting guide 250-270 each have a distal end which extends at about a 45 degree angle relative to the body or shaft of the cutting guide, in the remembered state. The cutting guides 250-270 are also illustrated having different diameters. For example, cutting guide 250 is a D5-45 (e.g., a 5 mm diameter cutting guide with a 45 degree bend at the distal end). Again, the distal end may be any length (e.g., 5 mm, 10 mm, and 15 mm lengths are illustrated).

It is noted that the cutting guides are not limited to having any particular angle, and may have a bend which is greater than and/or less than the illustrated 45 and 90 degrees. In some examples, the cutting guide 200 may have a straight or substantially straight side-profile in the remembered state (e.g., 0 degrees relative to the body or shaft).

FIG. 3 illustrates example flexible shaft cutting tips or “drill bits” 300 (individually referenced as 310-330) which may be implemented with the medical device 10. The device 10 using flexible cutting blades 300 enable a smooth forward or insertion movement, with cutting action occurring when pulled outward. The blades may be varying diameter to assist with the creation of cylindrical cavities within vertebral bodies and across the disc space to adjacent vertebral bodies. In FIG. 3, the cutting tips 300 are shown having a cutting edge, and may be inserted through a cutting guide (e.g., the cutting guides 200 illustrated in FIG. 2).

Again, different bends are shown in FIG. 3 for purposes of illustration. For example, cutting tip 310 is referred to as an E0 tip having a distal end extending at 0 degrees relative to the body or shaft; cutting tip 320 is referred to as an E45 tip has a distal end extending at 45 degrees relative to the body or shaft; and cutting tip 330 is referred to as an E90 tip having a distal end extending at 90 degrees relative to the body or shaft. It is noted, however, that flexible shaft cutting tips 300 may have any suitable bend greater than and/or less than the bends illustrated in FIG. 3.

Cutting tips 300 may be selected for use with a cutting guide 200 having a similar profile (e.g., an E45 tip may be selected for use with a Dx45 cutting guide). The outer diameter of the cutting tips may be sized to fit within a larger diameter cutting guide.

During use, the cutting tips may be operated to spin and advance (e.g., manually and/or automatically such as by a motor). When spinning and advancing into vertebrae, cutting tips 300 may core out a cylinder of bone. In some examples, cutting tips 300 may be hollow, allowing for application of suction to remove bone debris (e.g., from cutting or drilling).

FIG. 4 illustrates example flexible shaft balloon catheters 400 (individually referenced as 410-430) which may be implemented with the medical device 10. The balloon catheters 400 may be sized for receipt within the internal lumen of the cannula and trocar assembly shown in FIG. 1.

Catheter 410 is referred to as an F9 catheter, and is illustrated having a balloon tip 412 extending at 0 degrees; catheter 420 is referred to as an F45 catheter, and is illustrated having a balloon tip 422 extending at 45 degrees; and catheter 430 is referred to as an F90 catheter, and is illustrated having a balloon tip 432 extending at 90 degrees. These angles are relative to the body or shaft of the catheter, and are shown merely for purposes of illustration. Other angles may also be provided.

The length of the distal end of the catheter (e.g., the portion having the balloon) may also vary according to application (e.g., from about 1 mm to 4 mm in some examples). Of course, other lengths may be less than and/or greater than these examples.

The balloons of may be made of any suitable material and provided in any of a variety of shapes including but not limited to spherical, hemi-spherical and dumbbell shaped. Balloon catheters 410, 420 and 430 may each have a proximal end with control means for selectively expanding and contracting balloons 412, 422, and 432 (respectively). In an example, operation of the balloon portion may be used to press biological material outwards to increase a cavity size.

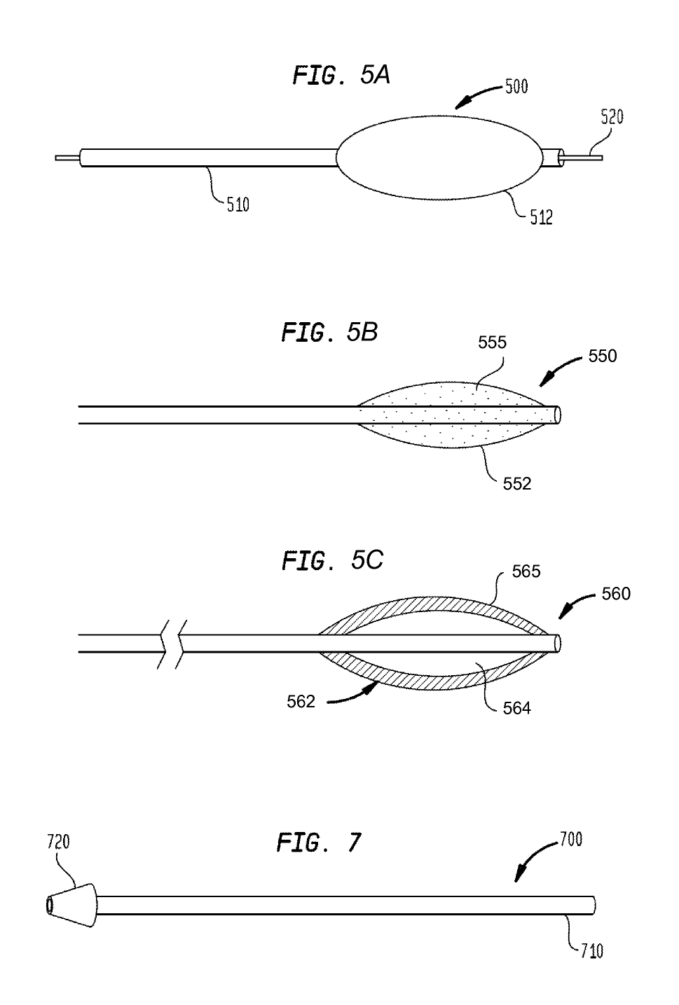

FIG. 5A illustrates another example of a flexible shaft catheter 500 having a balloon 512. The catheter 500 may have a hollow center that can be threaded on a guide wire into the vertebral body and then used to create a columnar cavity within the vertebral body. In an example, the catheter 500 may also function as a flexible cutting blade or “drill bit” with the balloon 512 used to vary the diameter of the cutting surface.

In an example, the shaft 510 of catheter 500 may be hollow, enabling a guide wire 520 to pass through in advance of the balloon 512. The guide wire 520 may include a cutting edge to cut a pathway of sufficient diameter to accommodate the balloon 512 of the catheter 500. The balloon 512 can then be inflated or otherwise expanded to facilitate further opening of the cavity.

FIGS. 5B-C illustrate other examples of a flexible shaft catheter having a balloon tip. The balloon may be housed by cutting blades that cut or “drill” a cylindrical cavity in the vertebral body as the shaft is spun. The diameter of the vertebral cavity can be varied by expanding or contracting the balloon.

The balloon is shown in FIGS. 5B-C as it may include outer surfaces configured with an abrasive. Accordingly, the balloon can be used in place of the cutting tips 300 shown in FIG. 3 and/or in place of the balloon catheter 400 and 500 shown in FIGS. 4 and 5A, respectively. In some examples, the size of the balloon may be expanded during cutting to progressively increase the size of the cavity.

In an example, the outer surface 552 of the balloon 550 may be entirely coated with an abrasive 555, as shown in FIG. 5B. In another example, the outer surface 562 of the balloon 560 may be partially coated and/or coated in sections (e.g., coated section(s) 565 and uncoated section(s) 564), as shown in FIG. 5C, e.g., to provide a number of cutting “blades.”

It is noted that any number and/or configuration (e.g., longitudinal, circumferential, spiral) of the blades may be provided. Any abrasive material may be provided on the outer surface of the balloon, including but not limited to diamond or carbon particles.

It is also noted that the balloon may be constructed from any of a variety of flexible materials, thereby enabling the balloon and associated cutting surfaces to expand and contract to a variety of sizes. For example, in a smaller size, balloon may be readily inserted through a cannula or other guide, and expanded to create and/or enlarge a cavity in a bone structure and/or other biological tissue.

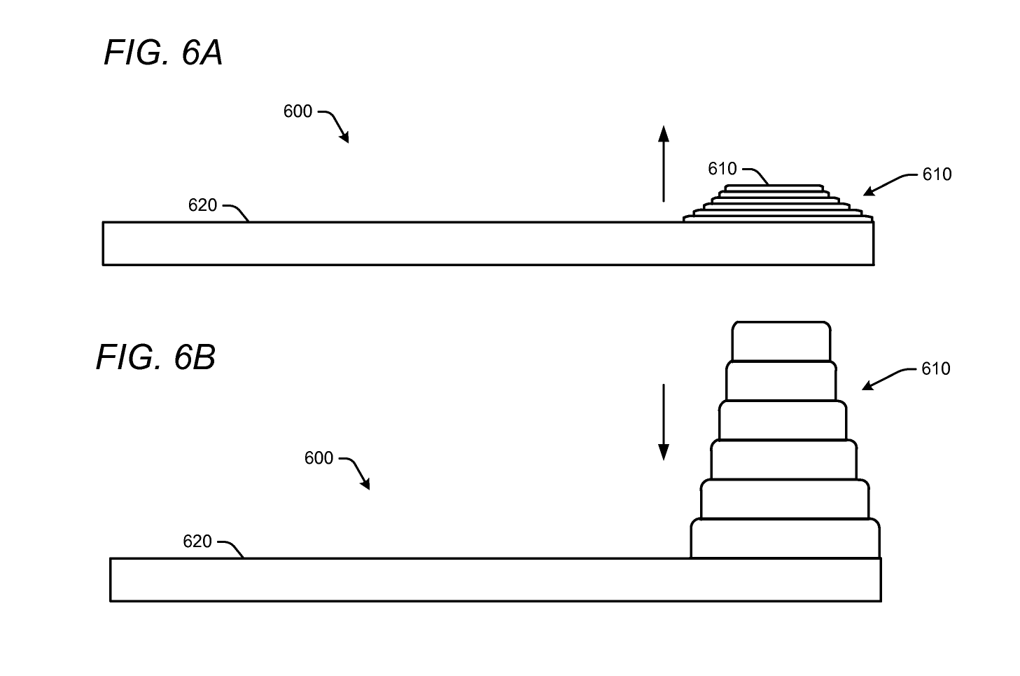

FIGS. 6A-B illustrate another example cutting tip 600 which may be used with the medical device. The cutting tip 600 can be provided in a closed or collapsed state (e.g., FIG. 6A) for insertion through the transpedicular cannula. The cutting tip 600 includes a vertical extensible cutting edge 610. Once positioned, the vertical extensible cutting edge 610 can be expanded (e.g., similar to expanding segments of an extendable radio antenna) to an expanded state (e.g., FIG. 6B). The cutting edge(s) 610 may be operated to rotate or spin to carve out a cylindrical cavity extending across one or more disc spaces, e.g., to form a cavity spanning multiple vertebra. In an example, the shaft 620 may be rectangular (or any desired shape). Also in an example, the shaft 620 may be configured to carry wire(s) operable to cause rotation of the cutting edge(s) 610.

FIG. 7 illustrates an example flexible shaft suction tubing 700 which may be implemented with the medical device 10. Flexible shaft suction tubing 700 is receivable within the internal lumen of a cannula (e.g., cannula and trocar assembly shown in FIG. 1). The suction tubing 700 may have an opening 710 on one end that forms an internal lumen through the shaft, and includes a connector 720 that can be coupled to a surgical suction source for removal of biological debris resulting from cutting or drilling.

FIG. 8 illustrates example flexible shaft probes 800 (individually referenced as 810-830) which may be implemented with the medical device 10. For example, the probes 800 may be inserted through the flexible NITINOL™ tubes and serve as a guide wire for a flexible cutting shaft. Probe 810 is referred to as an H0 probe, and is illustrated having a ball-tip probe 812 extending at 0 degrees; probe 820 is referred to as an H45 probe, and is illustrated having a ball-tip probe 822 extending at 45 degrees; and probe 830 is referred to as an H90 probe, and is illustrated having a ball-tip probe 832 extending at 90 degrees. These angles are relative to the body or shaft of the catheter, and are shown merely for purposes of illustration. Other angles may also be provided.

The length of the distal end of the probe may also vary according to application. Of course, other lengths may be less than and/or greater than these examples.

The probes 800 are receivable within the internal lumen of a cannula (e.g., cannula and trocar assembly shown in FIG. 1). In an example, the probes 800 may include an exterior insulating layer and an internal electrically conductive wire operatively coupled to the ball-tip probe. Application of an electrical current to a contact (e.g., contacts 814, 824, and 834) at the probe proximal end energizes ball-tip probes (e.g., 812, 822, and 832, respectively) so that energy can be applied to a subject material, such as bone or other biological tissue.

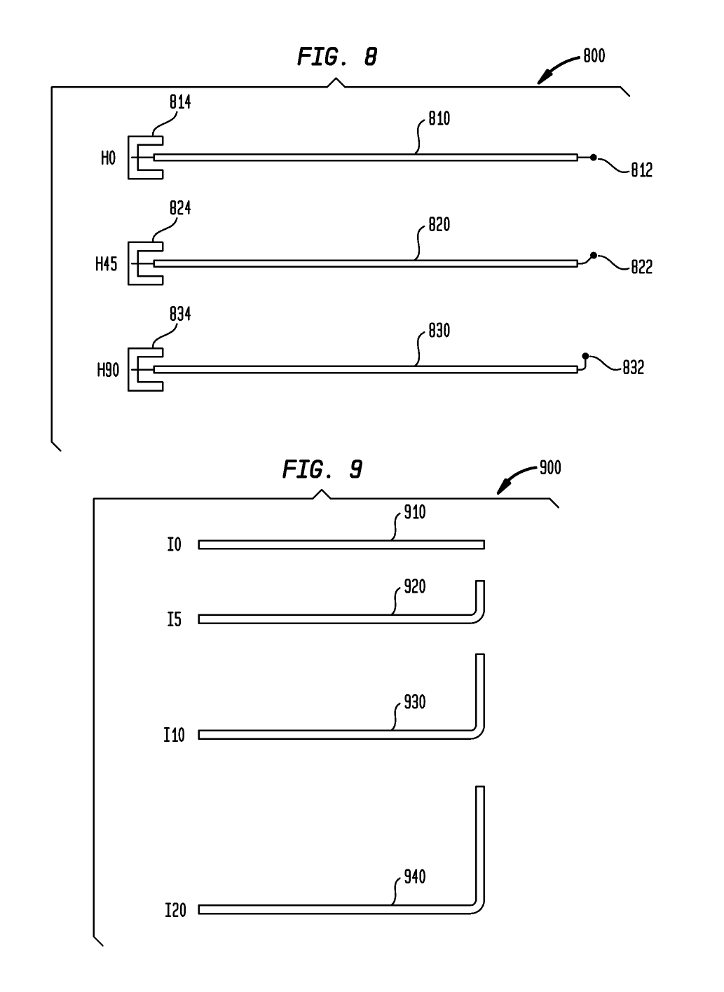

FIG. 9 illustrates example fill tubes 900 (individually referenced as 910-940) which may be implemented with the medical device 10. Tube 910 is referred to as an I0 tube, and is illustrated having a distal end extending at 0 degrees; tube 920 is referred to as an I5 tube, and is illustrated having a distal end extending 5 mm at 90 degrees; tube 930 is referred to as an I10 probe, and is illustrated having distal end extending 10 mm at 90 degrees; and tube 940 is referred to as an I20 probe, and is illustrated having distal end extending 20 mm at 90 degrees. The 90 angle is relative to the body or shaft of the catheter, and is shown merely for purposes of illustration. Other angles may also be provided. Likewise, the length of the distal end may also vary according to application. Other lengths may be less than and/or greater than these examples.

The flexible fill tubes 900 are receivable within the internal lumen of a cannula (e.g., cannula and trocar assembly in FIG. 1), and may be configured to transport and/or deliver filler or cement, biological agents, and/or other substances into cavities formed in the vertebral bodies.

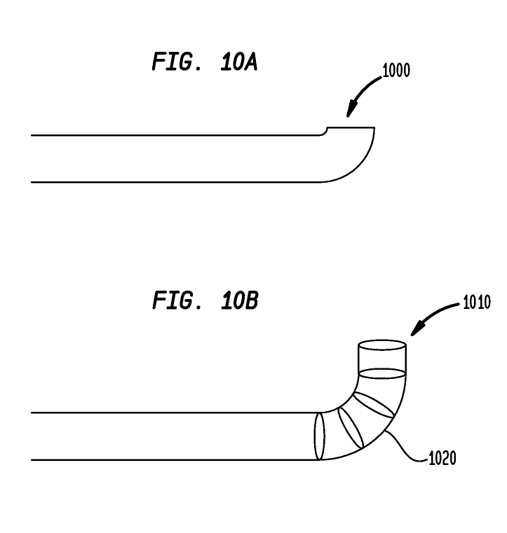

FIGS. 10A-B illustrate other example catheter tips which may be provided on any of the trocar, cannula, catheter, tubing, probes, and/or other bodies described herein, to provide direction. The catheter tip 1000 is shown in FIG. 10A as it may be configured with a distal end having a fixed angle (e.g., 90 degrees). Other fixed angles are also contemplated.

The catheter tip 1010 is shown in FIG. 10B as it may be configured with a distal end having a variable angle tip (including no angle). In an example, a deformable interface 1020 may be provided as part of a unitary structure enabling flexion. For example, segments with a large outer diameter cross section may be interconnected by smaller diameter cross section segments to enable flexion. In another example, movable distal end may be manufactured of separate components hinged or otherwise linked together to enable movement of the distal end relative to proximal end. Thus, the distal end may be configured to change into different angles. Any suitable materials for the interface 950 may be used.

Before continuing, it should be noted that the examples of medical device 10 and components thereof shown in the drawings and described above are provided for purposes of illustration, and are not intended to be limiting. Other devices and/or device configurations may be utilized to carry out the operations described herein. Having described the medical device 10 and components thereof according to a variety of illustrative examples, operation of the medical device 10 for a spinal surgery will be described with reference to the remaining drawings.

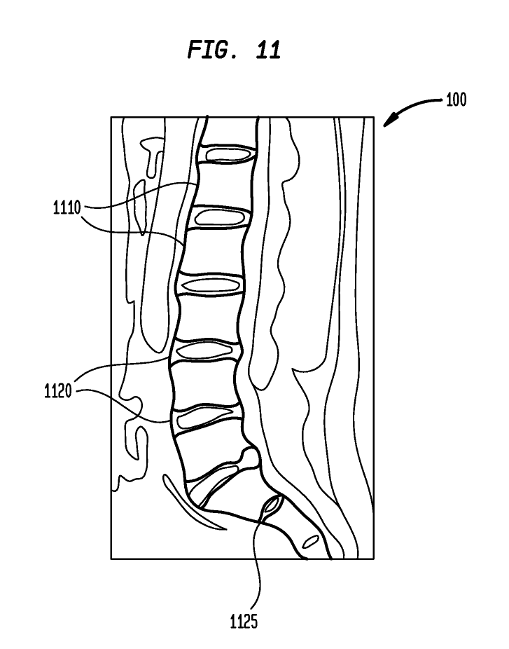

The following figures illustrate example endovertebral spinal surgery techniques. FIG. 11 illustrates a cross-section of a portion of a human spine, showing a vertebral body 1100, having vertebrae 1110, healthy discs 1120, and a herniated disk 1125. In an example surgical procedure, a cannula and trocar assembly of the medical device 10 may be inserted into the pedicle from a posterior approach under fluoroscopic guidance. In an example, the cannula and trocar assembly is inserted into the pedicle percutaneously.

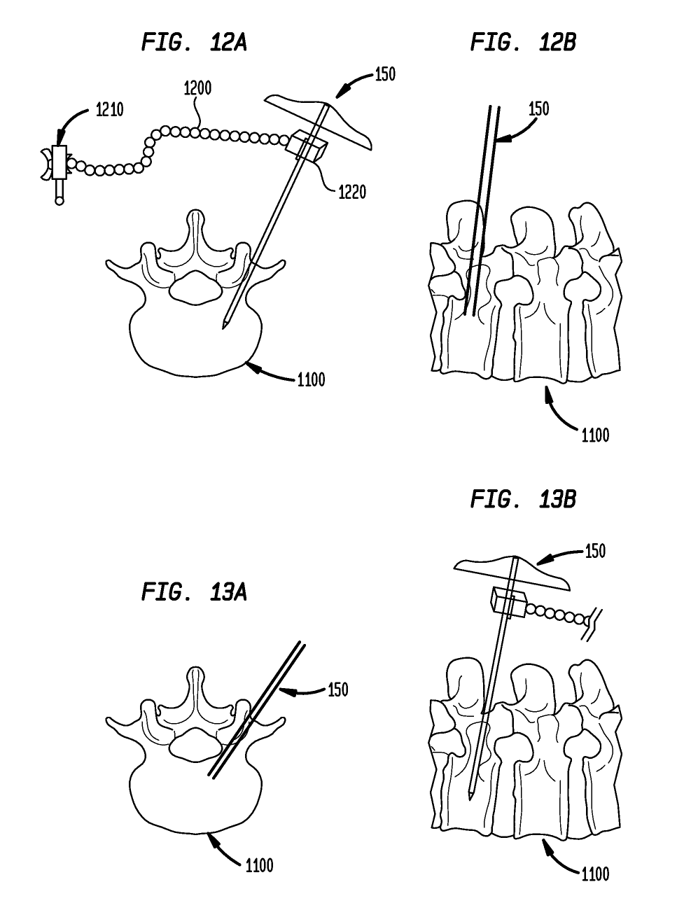

FIGS. 12A-B and FIGS. 13A-B illustrate side and top views of the medical device 10 during endovertebral spinal surgery. In an example, the medical device 10 includes an articulating arm or flexible snake 1200. The flexible snake 1200 may have a first end 1210 configured for gripping or mounting to a support (e.g., a surgical table or other stand). The flexible snake 1200 may also have a second end 1220 configured to securely grasp a cannula and trocar assembly of the medical device 10.

In an example, the first end 1210 may include a receptacle for receiving a support rod and a wing screw or bolt for compressing the support rod within the receptacle. The second end 1220 may include a receptacle and wing fastener configured for compressing the cannula and trocar assembly within the second end receptacle. As such, the flexible snake 1200 may be secured at one end (e.g., to an operating room table or other supportive object) by a locking mechanism. Flexible snake 1200 can then be used to firmly hold cannula and trocar assembly to facilitate precise percutaneous insertion of the cannula and trocar assembly through the pedicle and into a vertebral body 1100.

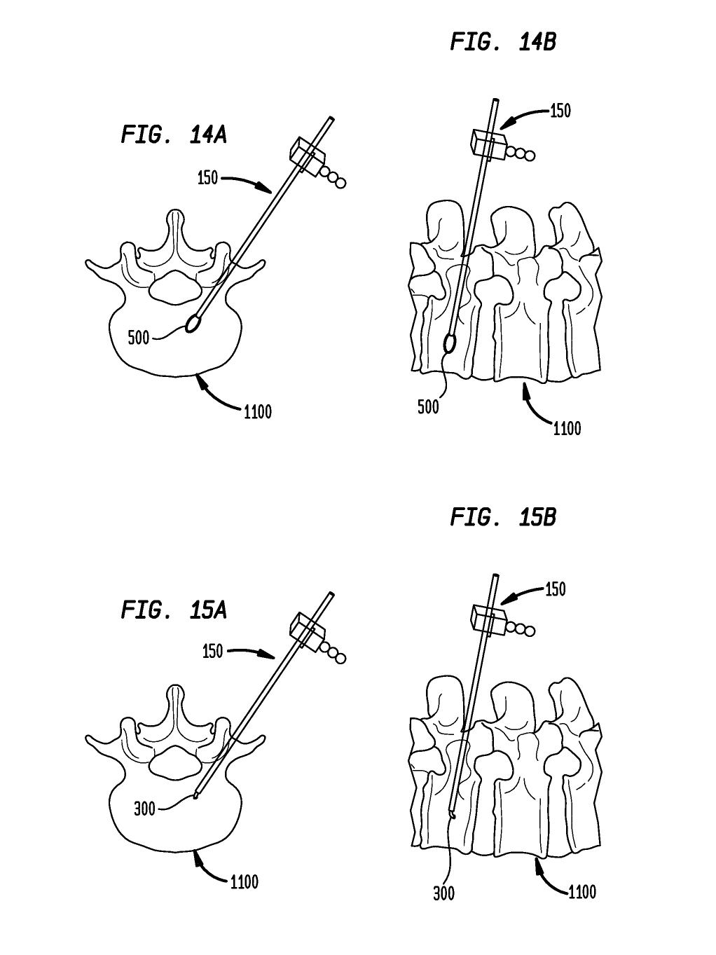

FIGS. 14A-B illustrate side and top views of example insertion of a balloon catheter 500 through the cannula and trocar assembly and into vertebrae 1100. The balloon may be operated to create a cavity in the vertebrae area. For example, balloon catheter 500 may be expanded to create a space within vertebral body 1100. This space can then be used to introduce a cutting guide 300 as shown in FIGS. 15A-B.

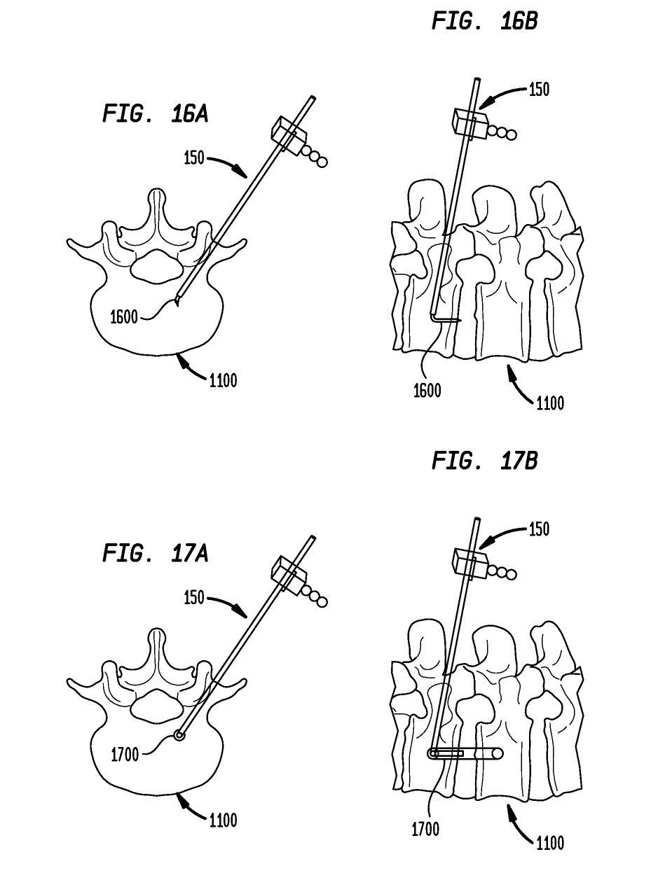

FIGS. 15A-B illustrate side and top views of example insertion of cutting guide 300 into vertebral body 1100. FIGS. 16A-B illustrate side and top views of example flexible shaft cutting tips 1600 (including but not limited to one or more of the cutting tips 400 shown in FIG. 4 and/or the balloons shown in FIGS. 6A-B) passing through cutting guide 310 which can be received within cannula and trocar assembly. FIGS. 17A-B illustrate side and top views of example use of a medical device 15 to core out a cylinder in the vertebral bodies 1100 and across disc space. In an example, a cutting tip 1600 (including but not limited to one or more of the cutting tips 300 shown in FIG. 3 and/or the balloons shown in FIGS. 6A-B) is rotated to form the cavity.

In an example, a wire with a diamond or cutting tip, or catheter with a central canal for a guide wire and cutting tip and expansive abrasive balloon, can be inserted into the trocar and spun either by hand or by machine to create a larger cavity. The curved trocar is turned so that the wire advanced through the needle is passed superiorly through the disc space above and, if necessary, through the rostral endplate and into the rostral vertebral body itself.

The wire (or catheter) with cutting tip may be spun as it is advanced using a manual or mechanized (e.g., partially or fully automated) device such that a cylinder of definable diameter is cored out of the vertebrae and disc space as the wire is advanced superiorly. A flexible cutting tip can then be inserted over and advanced along the guidewire. With its variable diameter tip, flexible cutting tip enables a technician or surgeon to ream a shaft of variable angle in the required direction through disc space and into an adjacent, for example rostral, vertebra.

After forming a cavity in the vertebra, a balloon catheter may be used to open up the cavity and/or a suction catheter (e.g., catheter 700 shown in FIG. 7) may be used to clean out the cavity before adding a material to the cavity. A probe (e.g., probe 800 shown in FIG. 8) may also be used to deliver electrical and/or radiative energy to the site.

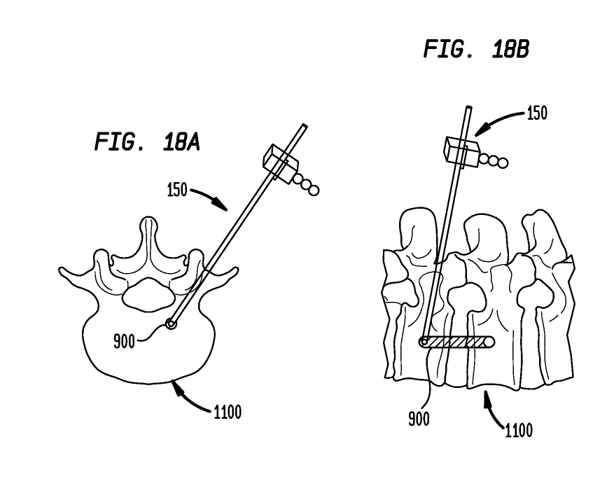

A flexible fill tube (e.g., tube 900 shown in FIG. 9) may then be inserted into the cannula and trocar assembly to provide a material such as a cement or filler, biological agent, and/or other material to the cavity. FIGS. 18A-B illustrate example filling of cavity. Cavity can be fully or partially filled with material to alter the dynamics of motion between vertebrae 2000 and 4000 from fusion (non-motion) and varying degrees of elasticity).

In an example a balloon can be filled with cement which hardens and thereby secures the two vertebral bodies. In another example, the cavity can be filled directly with cement to fix the two vertebral bodies without the use of a balloon.

Example biological agents may include but are not limited to allograft and/or autograft bone slurry configured to induce fusion between adjacent vertebrae and biological materials configured to emulate the modulus of elasticity of normal intervertebral disc material. Example filling agents may include but are not limited to, cement, grout or paste such as poly(methyl methacrylate) (PMMA). Substances of varying flexibility may be used as filling agents.

The operation of the medical device and components thereof which are shown and described herein are provided to illustrate example operation. It is noted that operation of the medical device is not limited to the techniques or ordering shown and described. Still other operations may also be implemented.

It is noted that a balloon can be inflated in the caudal (lower, or “near the tail” of the person) vertebra and then inflated again at the top of a cored out cylinder, extending into one or more vertebra above cylindrical cavity. This creates create a “dumbbell” shaped cavity which, when filled with a filling agent to anchor the two ends and modulate kinetics between the vertebrae.

In some example techniques, application of a vacuum may draw disc material back into the disc space and away from nerve roots and spinal cord. The cylindrical cavity extending between two vertebrae can then be filled with a substance that expands within a balloon to distract two more vertebrae.

It is noted that the examples shown and described are provided for purposes of illustration and are not intended to be limiting. Still other examples are also contemplated.

1. A percutaneous transpedicular endovertebral device, comprising:
a cannula having an internal lumen;
a trocar receivable within the cannula internal lumen;
a flexible shaft cutting catheter receivable through the trocar to form a cavity within a vertebrae structure; and
a delivery tube receivable through the trocar to deliver filling, biological agent, or other material to the cavity.

2. The device of claim 1, further comprising a balloon catheter.

3. The device of claim 1, wherein the at least one trocar assumes a straight profile in a distorted shape and a curved profile in a remembered shape.

4. The device of claim 1 wherein the at least one flexible shaft cutting catheter includes a guide wire with a cutting portion.

5. The device of claim 1, wherein the at least one flexible shaft cutting catheter includes a balloon cutter.

6. The device of claim 5, wherein the balloon cutter has an abrasive exterior surface.

7. The device of claim 5, wherein the balloon cutter has an expandable cutting blade.

8. The device of claim 7, wherein the cutting blade expands upon inflation of a balloon portion and the cutting blade retracts upon deflation of the balloon portion.

9. The device of claim 1, further comprising a variable angle tip catheter shaft receivable through the trocar.

10. The device of claim 1, wherein the trocar has a tapered threaded portion to mount a cannula and trocar assembly to a bone structure.

11. The device of claim 1, further comprising a vertical extensible cutting edge having segments foldable between a collapsed state and an expanded state, the cutting edge operable in the expanded state to cut a cylindrical cavity extending across one or more disc spaces to span multiple vertebra.

12. A percutaneous transpedicular endovertebral system, comprising:
at least one cannula having an internal lumen;
at least one trocar receivable through the internal lumen of the cannula;
a plurality of flexible shaft cutting guides each having a different profile, the flexible shaft cutting guides receivable through the at least one trocar;
a plurality of flexible shaft cutting shafts each having a different profile, the flexible shaft cutting shafts receivable through the at least one trocar;
at least one flexible shaft suction tubing receivable through the at least one trocar; and
at least one flexible delivery tube receivable through the at least one trocar.

13. The system of claim 12, further comprising an articulating arm having a first end configured to mount on a support structure, and a second end configured to securely grasp the cannula.

14. The system of claim 12, wherein the flexible shaft cutting guides have an angled distal end deformable to fit through the at least one trocar, and automatically returning to a non-deformed configuration upon exiting the at least one trocar.

15. The system of claim 12, further comprising a flexible shaft ball-tipped probe, the flexible shaft ball-tipped probe has an exterior insulating layer around an electrically conductive wire operatively coupled to a ball tip.

16. The system of claim 12, further comprising a fixed-tip catheter receivable through the at least one trocar to guide at least one of the flexible shaft cutting guides, the flexible shaft cutting shafts, the at least one flexible shaft suction tubing, and the at least one flexible delivery tube.

17. The system of claim 12, further comprising a flexible tip catheter receivable through the at least one trocar to guide at least one of the flexible shaft cutting guides, the flexible shaft cutting shafts, the at least one flexible shaft suction tubing, and the at least one flexible delivery tube.

18. A method of providing a percutaneous transpedicular endovertebral device, comprising:
providing a trocar and a cannula for percutaneous access to a vertebral structure;
providing a guide for a flexible shaft cutting shaft through the trocar to form a cavity in the vertebral structure; and
providing structure for delivering a filling, biological agent, or other material to the cavity.

19. The method of claim 18, further comprising translating the flexible shaft cutting end from an angled profile to a straight profile as the flexible shaft cutting end moves through the trocar, and translating the flexible shaft cutting end from the straight profile back to the angled profile as the flexible shaft cutting end exits a distal end of the trocar.

20. The method as set forth in claim 18, further comprising providing an inflatable balloon.
